(19) United States
(12) Patent Application Publication
(10) Pub. No.: US 2022/0202165 A1
(43) Pub. Date: Jun. 30, 2022

(54) SINGLE-USE TRAY FOR ADMINISTERING A SPA TREATMENT

(71) Applicant: Millenia Productions, LLC, Wichita, KS (US)

(72) Inventor: Michael Nghiem Le, Wichita, KS (US)

(73) Assignee: Millenia Productions, LLC, Wichita, KS (US)

(21) Appl. No.: 17/565,810

(22) Filed: Dec. 30, 2021

Related U.S. Application Data
(60) Provisional application No. 63/133,200, filed on Dec. 31, 2020.

(51) Int. Cl. A45D 40/24; A61Q 19/00; B65D 1/36; B65D 77/20; A61K 8/02; A61K 8/65

(52) U.S. Cl. CPC A45D 40/24; A61Q 19/00; B65D 1/36; B65D 77/2024; A61K 2800/88; A61K 8/65; A61K 2800/28; A45D 2200/25; A61K 8/0212

(57) ABSTRACT

A disposable manicure kit includes a single-use plastic tray that presents a plurality of receptacles that are suitable for receiving spa treatment products. The tray presents a generally flat upper surface between and around the receptacles. The tray and its receptacles is sealed by removable covering. The covering has an upper surface for displaying product and product use related indicia. The removable covering is adapted for sealing with the upper surface of the tray in such a manner as to seal each receptacle and isolate the contents of each receptacle from the contents of the other receptacles. A plurality of spa products are deposited separately such that each spa product is disposed in a corresponding one of the plurality of receptacles and then sealed by the removable covering. After removal of the covering, the spa products can be used in a predetermined sequential manner to a series of spa treatments.

### CROSS-REFERENCE TO RELATED APPLICATIONS

This application claims the benefit of provisional patent application No. 63/133,200 filed on Dec. 31, 2020, which is incorporated herein as if repeated verbatim hereinafter.

### FIELD

Embodiments of the invention relate to containers for housing one or more spa products that collectively form a spa treatment. More particularly, embodiments of the invention relate to housing single-use amounts of one or more spa products (e.g., products that may be used for administering a manicure treatment and/or a pedicure treatment) in separate receptacles of a single treatment tray.

### BACKGROUND

Skin care products are often used in providing manicure and pedicure, or other beauty or skin-care services. While there is a myriad of skin care products available in the market like those that may be used in the various steps of a beauty or skin-care treatment, these skincare products often are sold separately and in bulk or multi-use packaging.

This bulk packaging, often in five-gallon containers, may be unsightly and take up limited space in a spa or salon, and at the same time may be unsuitable to provide display or point-of-sale advertisement of the service provided. Further, the application of beauty and skincare products from such multi-use packaging may be imprecise, as there may not be a way to administer a consistent application of product to each user. Each technician may have his or her own idea of how much beauty or skincare product is appropriate for an application for each given user. This may make it difficult for a service provider to provide a standardized and consistent beauty or skincare treatment to its customers.

Bulk or multi-service packaging also may make it more difficult for a service provider to keep track of its inventory when it may not be sure how many individual applications may be found in the multi-use packaging. A service provider also may have to make large initial investments in bulk multi-use packaging of beauty and skin-care products, and thus, may also be hesitant to make similar investments to try new products and services that also may require up-front costs.

Multi-use packaging also affects how sanitary, effective, or pleasing to the senses a beauty or skincare product may be. In bulk packaging, the skincare or beauty product may become contaminated from repeated contact with technicians or users and the environment as a result of being accessed many times as a technician accesses the contents of the multi-use packaging on a repeated basis. Beauty and skincare products stored in multi-use packaging may also suffer from a reduced shelf-life due to repeated exposure to the environment each time a technician accesses the contents of the multi-use packaging. The beauty or skincare product may accordingly also suffer from reduced quality and diminished aroma.

As such skincare products often are sold not only in bulk or multi-use packaging, but the individual products that collectively comprise a single spa treatment may be sold separately. Accordingly, each technician may not only need to determine an amount of product to use but also which products to use and in what order to affect a particular treatment. This introduces uncertainty and inconsistency into customers' experiences with the service provider.

### SUMMARY

In accordance with the disclosed subject matter, a single use tray for providing a manicure or pedicure treatment, or other beauty or skincare treatment, is provided. The single use tray may include a tray portion having one or more receptacles formed therein, each receptacle configured for receiving a predetermined amount of an individual spa product. A plurality of individual spa products housed within a particular tray portion may collectively form a spa treatment. In embodiments, such a spa treatment may include a plurality of sequentially administered spa products, the number comprising the plurality being equivalent to the number of receptacles formed in the tray portion of the single use tray.

In embodiments, the single use tray may include a lid portion. Each of the tray portion and the lid portion may include respective features for permitting the temporary adhesion of the lid portion and the tray portion, via any suitable, temporary adhesive known to those having ordinary skill in the art. Each of the tray portion and the lid portion further may include respective features for permitting the removal of the lid portion from the tray portion by means of adhesive disengagement. In embodiments, the lid portion may include a flange configured to make it easier for a user to grip the lid portion for removal thereof. In embodiments, the lid portion, when adhered to the tray portion, may form a substantially horizontal surface suitable for stacking multiple single use trays upon one another for storage and/or display. The outwardly facing surface of the lid portion may contain an artistic design believed to be pleasing to a user and/or a customer of a user, an identity of the spa treatment housed inside and/or one or more of the spa products comprising the housed spa treatment, instructions for use of the spa treatment and/or one or more spa products housed inside, or the like.

In accordance with the disclosed subject matter, if desired, the lid portion of the single-use tray may be discarded upon removal. In embodiments, subsequent to administration of the one or more individual spa products housed within the receptacles of the tray portion of the single-use tray, the entire single-use tray may be discarded, if desired.

There has thus been outlined, rather broadly, the features of the disclosed subject matter in order that the detailed description thereof that follows may be better understood, and in order that the present contribution to the art may be better appreciated. There are, of course, additional features of the disclosed subject matter that will be described herein after and which will form the subject matter of the claims appended hereto.

In this respect, before explaining at least one embodiment of the disclosed subject matter in detail, it is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures for carrying out the several purposes of the disclosed subject matter. It is important, therefore, that the claims be regarded as including such equivalent constructions insofar as they do not depart from the spirit and scope of the disclosed subject matter.

These together with the other objects of the disclosed subject matter, along with the various features of novelty which characterize the disclosed subject matter, are pointed out with particularity in the claims appended to and forming a part of this disclosure. For a better understanding of the disclosed subject matter, its operating advantages and the specific objects attained by its uses, reference should be had to the accompanying drawings and descriptive matter in which there are illustrated various embodiments of the disclosed subject matter.

### BRIEF DESCRIPTION OF THE DRAWINGS

The accompanying drawings, which are included to provide a further understanding of the disclosure, are incorporated in and constitute a part of this specification, illustrate embodiments of the disclosure and, together with the Detailed Description, serve to explain the principles of the disclosure. No attempt is made to show structural details of the disclosure in more detail than may be necessary for a fundamental understanding of the disclosure and the various ways in which it may be practiced. The drawings are not necessarily to scale, emphasis instead being placed upon illustrating the principles of the invention. Features of one embodiment may be employed with other embodiments as the skilled artisan would recognize, even if not explicitly stated herein. Descriptions of well-known components, processing, and techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. In the drawings:

FIG. 1 is a diagram illustrating a top view of a single-use tray for administering a spa treatment, in accordance with embodiments of the present disclosure.

FIG. 2 is a diagram illustrating a perspective view of a single-use tray for use in accordance with some embodiments of the disclosure, the single-use tray having a lid portion adhered thereto.

FIG. 3 is a diagram illustrating a perspective view of the single-use tray of FIG. 2 with the lid portion removed exposing a plurality of spa products contained within the receptacles of the single-use tray, in accordance with some embodiments of the present disclosure.

FIG. 4 is a diagram illustrating a side view of the single-use tray of FIG. 2 except in FIG. 4 the lid portion is attached.

### DETAILED DESCRIPTION

The foregoing and other aspects of the present invention will now be described in more detail with respect to various embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the present disclosure is for the purpose of describing particular embodiments only and is not intended to be limiting of embodiments of the disclosed invention. As used in the present disclosure, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention pertains. It will be further understood that terms, such as those defined in commonly used dictionaries, should be interpreted as having a meaning that is consistent with their meaning in the context of the present disclosure and relevant art and should not be interpreted in an idealized or overly formal sense unless expressly so defined herein. In case of a conflict in terminology, the present specification is controlling.

As used herein, “and/or” refers to and encompasses any and all possible combinations of one or more of the associated listed items, as well as the lack of combinations when interpreted in the alternative (“or”).

Unless the context indicates otherwise, it is specifically intended that various features of the present disclosure can be used in any combination. Moreover, the present disclosure also contemplates that in some embodiments, any feature or combination of features set forth herein can be excluded or omitted. To illustrate, if the specification states that a product comprises components A, B and C, it is specifically intended that any of A, B or C, or a combination thereof, can be omitted and disclaimed.

The terms “including,” “comprising,” and variations thereof, as used in this disclosure, mean “including, but not limited to,” unless expressly specified otherwise.

As used in this disclosure, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U. S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value, such as, for example, an amount or concentration, is meant to encompass variations of ±20%, ±10%, ±5%, ±1%, ±0.5%, or even ±0.1% of the specified amount. A range provided herein for a measurable value may include any other range and/or individual value therein.

The term “disposable,” as used herein, refers to an article designed to be discarded after use. As such, in accordance with this disclosure, some embodiments of the single-use tray described herein are designed to be disposable, that is, designed to be used to provide a spa treatment to a single individual, the spa treatment including one or more of the spa products housed inside of the receptacles in the tray prior to use, and then discarded.

As previously set forth, beauty or skin-care treatments and/or products often are sold in bulk or multi-use packaging. The application of beauty and skincare products from multi-use packaging may be imprecise, as there may not be a way to administer a consistent application of product to each user. Bulk or multi-service packaging also may make it more difficult for a service provider to keep track of its inventory when it may not be sure how many servings may be found in the multi-use packaging. Additionally, multi-use packaging also may affect how sanitary, effective, or pleasing to the senses a beauty or skincare product may be. In bulk packaging, the skincare or beauty product may become contaminated from repeated contact with technicians or users and the environment as a result of being accessed many times as a technician accesses the contents of the multi-use packaging on a repeated basis. As such skincare products often are sold not only in bulk or multi-use packaging, but the individual products that collectively comprise a single spa treatment may be sold separately. Accordingly, each technician may not only need to determine an amount of product to use but also which products to use and in what order to affect a particular treatment. This introduces uncertainty and inconsistency into customer's experiences with the service provider.

Embodiments of the present disclosure address these drawbacks of traditional spa treatment systems by providing a self-contained, single use system for administering a manicure treatment, a pedicure treatment, or any other beauty or skin-care service. The self-contained, single use system may include a tray including a tray portion with one or more receptacles formed therein, each receptacle configured for receiving a predetermined amount of an individual spa product. A plurality of individual spa products housed within a particular tray portion may collectively form a spa treatment. In embodiments, such a spa treatment may include a plurality of sequentially administered spa products, the number comprising the plurality being equivalent to the number of receptacles formed in the tray portion of the single use tray.

In embodiments, the single use tray may include a lid portion. The lid portion may be a thin, flat sheet of material sized and shaped to be adhered to the tray portion via any suitable, temporary adhesive known to those having ordinary skill in the art. In embodiments, the lid portion may be substantially flat and sized and shaped for adhesive engagement with the tray portion as pressure is applied to the tray portion and/or the lid portion, as is well known in the art. In embodiments, the lid portion may include a flange configured to make it easier for a user to grip the lid for removal thereof.

In embodiments, the lid portion, when adhered to the tray portion, may form a substantially horizontal surface suitable for stacking multiple single use trays upon one another for storage and/or display. The outwardly facing surface of the lid portion may contain an artistic design believed to be pleasing to a user and/or a customer of a user, an identity of the spa treatment housed inside and/or one or more of the spa products comprising the housed spa treatment, instructions for use of the spa treatment and/or one or more spa products housed inside, or the like.

In accordance with the disclosed subject matter, if desired, the lid portion of the single-use tray may be discarded upon removal. In embodiments, subsequent to administration of the one or more individual spa products housed within the receptacles of the tray portion of the single-use tray, the entire single-use tray may be discarded, if desired.

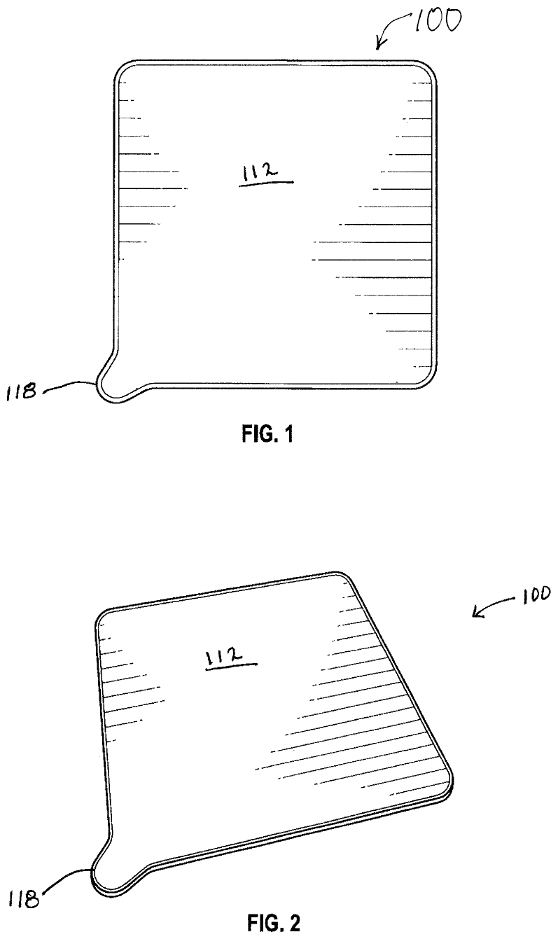

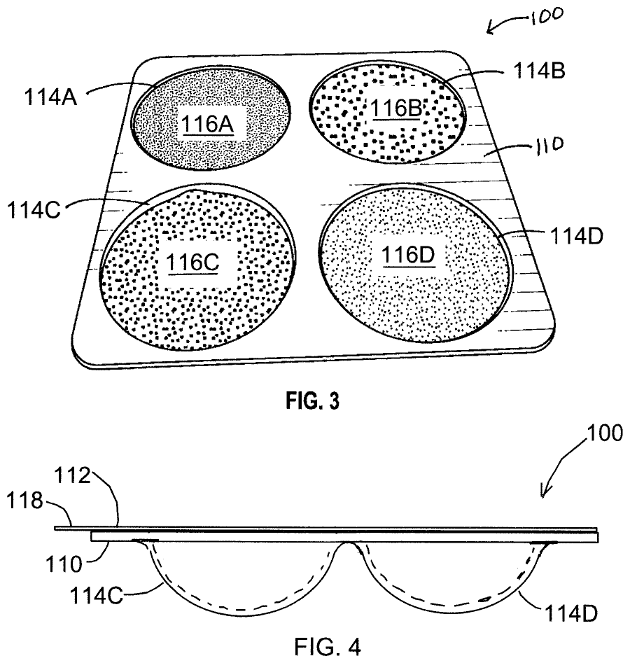

With reference now to FIGS. 1-4, illustrated is a single-use tray 100 for use according to some principles of the disclosure, the single-use tray 100 including a tray portion 110 and lid portion 112. Each of the tray portion 110 and a corresponding lid portion 112 may include respective features for permitting the temporary adhesion of the lid portion 112 and the tray portion 110 and removal of the lid portion 112 from the tray portion 110 by means of adhesive disengagement. The lid portion 112 of the single-use tray 100 may be substantially flat and sized and shaped for adhesive engagement with the tray portion 110 as pressure is applied between the lid portion 112 and the tray portion 110. In embodiments, the lid portion 112 may include a flange 118 configured to make it easier for a user to grip the lid portion 112 for removal thereof

The tray portion 110 may include one or more receptacles 114A, 114B, 114C and 114D. Each receptacle 114A-D is configured for receiving a predetermined amount of an individual spa products 116A, 116B, 116C and 116D. A plurality of individual spa products housed within a particular tray portion 110 may collectively form a spa treatment. In embodiments, such a spa treatment may include a plurality of sequentially administered spa products, the number comprising the plurality being equivalent to the number of receptacles such as receptacles 114A-D in this example, formed in the tray portion 110 of the single use tray 100.

In embodiments, the lid portion 112 may not be replaceable once removed from engagement with the tray portion 110. In other embodiments, the lid portion 112 may be replaced by re-adherence with the tray portion 110. In still other embodiments, a second lid portion (not shown) may be adhered to the tray portion 110 to cover the receptacles 114A-114D of the tray portion 110 once the originally adhered lid portion 112 has been removed.

By way of example, receptacle 114A may contain approximately 1 ounce of Soaking Crystals or soaking salts. Such soaking salts may be used to create a detoxifying bath made with natural sea salt that brings out impurities and may soothe the skin. Accordingly, in this example, the ingredients contained in receptacle 114A may include the following: powder sea rock, mineral rock, sodium bicarbonate, sodium chloride, chloramine T-sodium, sequicarbonate tripolyphosphate, fragrance (Parfum), maltodextrin, isopropyl palmitate, Oleamide DEA, Spearmint Oil (Mentha Spicata), dimethicone, aloe vera (Aloe Barbadensis), 0.1% CBD isolate, Vitamins E, and colorings. It is preferred that such soaking salts are dissolved into a basin of water as may be suitable for either for soaking hands or feet.

By way of example, receptacle 114B may contain approximately 1 ounce of a sugar cane scrub. Accordingly, in this example the ingredients contained in receptacle 114B may include the following: Aloe Vera (Barbados Aloe), Vitamin E (Tocopheryl Acetate), Magnesium Sulfate, Maltodextrin, Mineral Oils, Sugar (Evaporated Cane Juice), Fruit Extract, Propylene Glycol, Glycerin, .1% CBD Isolate, Vitamin C&D, Fragrance (Parfum), Plant Collagen and colorings. It is preferred that the skin of either the hands or feet is moistened and that the sugar cane scrub is massaged into the skin for 1 to 5 minutes and then rinsed off thoroughly.

By way of example, receptacle 114C may contain approximately 1 ounce of a collagen cream mask. Accordingly, in this example the ingredients contained in receptacle 114C may include the following: Water, sesame (Sesame Indicum) oil, Aloe Vera Gel (Barbados Aloe), Menthol, Eucalyptus Oil (Globulus Leaf Oil)*, Chamomile Extract (Roman Anthemis Nobilis), Glycerin, Propylene Clycol, Kaolin, Cetyl Aicohol, Ceteareth 20, 0.1% CBD Isolate, Vitamin E, (Tocopheryl Acetate), Fragrance (Parfum), plant collagen and colorings. It is preferred that the collagen cream maske be applied evenly to the skin, allowed to dry for 2 to 3 minutes and then, not rinsed, but wiped off with a damp towel.

By way of example, receptacle 114D may contain approximately 1 ounce of a collagen massage lotion. Accordingly, in this example the ingredients contained in receptacle 114D may include the following: Water, Sesame (Sesame Indicum) Seed Oil, Aloe Vera (Barbados Aloe) Gel, Menthol, Eucalyptus Oil (Globulus leaf Oil), Chamomile Extract (Roman Anthemis Nobilis), Glycerin, Kaolin, Cetyl Alcohol, Ceteareth 20, 0.1% CBD Isolate, Vitamin E, (Tocopheryl Acetate), Plant Collagen, Fragrance (Parfum), Limonene, Lilial, Linalool, Lyral and colorings. The collagen massage lotion may be massaged into the skin of the feet or hands.

It is preferred, in the example embodiment described above, that a treatments be performed such that the order given above is followed by the operator. Thus ingredients present in 114A are used first and so on until all of the ingredients in all of the receptacles have been used in the order given above. The above example embodiment is merely intended as an example of how the single-use tray 100 may be used for conducting spa treatments.

As described above, implementations of the present disclosure relate to a single use system for administering a spa treatment. Embodiments of the present disclosure have been described in relation to particular aspects, which are intended in all respects to be illustrative rather than restrictive. Alternative embodiments will become apparent to those of ordinary skill in the art to which the present disclosure pertains without departing from its scope.

From the foregoing, it will be seen that this disclosure is one well adapted to attain all the ends and objectives set forth above, together with other advantages which are obvious and inherent to the single-use system. It will be understood that certain features and sub-combinations are of utility and may be employed without reference to other features and sub-combinations. This is contemplated by and is within the scope of the claims.

It is to be understood that while certain forms of this disclosure have been illustrated and described, it is not limited thereto, except in so far as such limitations are included in the following claims and allowable equivalents thereof.

Having thus described the invention, what is claimed as new and desired to be secured by Letters Patent is:

1. A single-use kit for a manicure or a pedicure, comprising:
(a) a single-use plastic tray that presents a plurality of receptacles that are suitable for receiving spa treatment products, the tray presenting a generally flat upper surface between and around the receptacles,
(b) a removable covering for the tray, the covering having an upper surface for displaying product and product use related indicia, the removable covering being adapted for sealing with the upper surface of the tray in such a manner as to seal each receptacle and isolate the contents of each receptacle from the contents of other receptacles,
(c) a plurality of spa products deposited separately such that each spa product is deposited in one of a corresponding one of the plurality of receptacles and then sealed by the removable covering, such that after subsequent removal of the removable covering, the spa products in each receptacle can be used in a predetermined sequential manner to a series of spa treatments.

2. The disposable manicure kit of claim 1, wherein;
the tray is fashioned from injection molded plastic.

3. The single-use kit of claim 1, wherein;
the plurality of receptacles includes a receptacle that contains bath salt.

4. The single-use kit of claim 1, wherein;
the plurality of receptacles includes a receptacle that contains a sugar scrub.

5. The single-use kit of claim 1, wherein;
the plurality of receptacles includes a receptacle that contains a collagen cream mask.

6. The single-use kit of claim 1, wherein;
the plurality of receptacles includes a receptacle that contains a collagen massage lotion.

7. The single-use kit of claim 1, wherein;
the plurality of receptacles includes a first receptacle that contains bath salt, a second receptacle that contains a sugar scrub, a third receptacle that contains a collagen cream mask and a fourth receptacle that contains a collagen massage lotion.
